SEQUENCE LISTING (5 sequences)

SEQ ID NO: 1 - protein, 381 aa, Artificial Sequence
  PTX3 polypetide
MHLLAILFCALWSAVLAENSDDYDLMYVNLDNEIDNGLHPTEDPTPCACGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 2 - DNA, 22 bp, Artificial Sequence
  PTX3 forward primer
gggacaagctcttcatcatgct

SEQ ID NO: 3 - DNA, 17 bp, Artificial Sequence
  PTX3 reverse primer
gtcgtccgtggcttgca

SEQ ID NO: 4 - DNA, 24 bp, Artificial Sequence
  GAPDH forward primer
agcaatgcctcctgcaccaccaac

SEQ ID NO: 5 - DNA, 22 bp, Artificial Sequence
  GAPDH reverse primer
ccggaggggccatccacagtct

(19) United States
(12) Patent Application Publication
(10) Pub. No.: US 2013/0309695 A1
(43) Pub. Date: Nov. 21, 2013

(54) METHODS OF DIAGNOSING ASTHMA

(71) Applicant: Abdelilah Soussi Gounni, Winnipeg (CA)

(72) Inventor: Abdelilah Soussi Gounni, Winnipeg (CA)

(73) Assignee: UNIVERSITY OF MANITOBA, Winnipeg, MB (CA)

(21) Appl. No.: 13/701,458

(22) PCT Filed: May 31, 2011
(86) PCT No.: PCT/CA2011/000655

§ 371 Date: Jul. 8, 2013

Related U.S. Application Data
(60) Provisional application No. 61/349,979, filed on May 31, 2010.

(51) Int. Cl. G01N 33/68

(52) U.S. Cl. CPC G01N 33/6893
USPC 435/7.92

(57) ABSTRACT

A method of detecting a risk for or presence of asthma in a subject. The method comprises obtaining a biological sample comprising human airway smooth muscle cells from a subject and testing the biological sample for the presence of a pentraxin-3 (PTX3) polypeptide. The presence and/or quantity of PTX3 polypeptide in the biological sample correlates with the risk for or presence of asthma in the subject.

### TECHNICAL FIELD

The present invention relates to diagnostic methods for asthma. More particularly, the invention relates to methods for detecting a risk for and/or the presence of asthma in a subject.

### BACKGROUND

Allergic and chronic asthmas are inflammatory obstructive conditions of respiratory airways and are generally characterized by bronchial hyper-responsiveness (bronchospasm), reversible airway obstruction and infiltration by inflammatory cells, and in some cases, airway remodeling. Symptoms include dyspnea, chest tightness, coughing and wheezing. These classic symptoms are easily observed clinically, but overlap with a variety of other disorders that may be unrelated to lung inflammation or an allergic response. For example, wheezing may commonly be associated with brochiolitis, asthma attacks, chronic obstructive pulmonary disorder (COPD), pulmonary edema, vocal chord dysfunction, anaphylaxis, aspiration of foreign matter or other obstructions of the airways such as a tracheal tumor, surgical complications (e.g. lobectomy of lung), bronchial stenos or the like. Dyspnea may commonly be associated with obstructive lung diseases or disorders such as asthma, bronchitis, COPD, cystic fibrosis, emphysema, some parasite infections (e.g. hookworm), or disease of lung parenchyma or pleura (e.g. pneumonia, alveolitis, hypersensitivity pneumonitis, some cancers or the like). Coughing is a response to secretions or irritants in the breathing passages and respiratory tract, and may commonly be associated with respiratory tract infections, smoking, exposure to air pollution, gastroesophageal reflux disease (GERD), post-nasal drip, heart failure and even some medications (e.g. ACE inhibitors).

Clinical diagnosis of asthma generally includes the steps of asking detailed questions of a subject to develop a medical history, assessment of the subject's breathing patterns with standard lung function testing protocols that may additionally include airway challenge testing, chest x-rays, laboratory assessments of the subject's blood and/or sputum samples, epicutaneous allergy testing, and response to trial dosing with a selected asthma medication. However, no single indicator is definitive of asthma. When a subject presents at an acute care centre or hospital with breathing difficulties requiring immediate treatment, there may be no medical history available to refer to. While adults may be capable of discussing medical history or trigger events that may aid in diagnosis, young children or elderly subjects experiencing confusion may not be able to describe the experience. While testing of lung function may be performed, the results provide a measure of the airflow through the bronchi thus indicating the degree of obstruction of the airway, which may or may not be related to an asthmatic condition. Consequently, diagnoses are often delayed by the need to eliminate other respiratory disorders through a process of clinical elimination.

### SUMMARY OF THE INVENTION

The exemplary embodiments of the present invention relate to methods for a identifying a risk for or a presence of asthma in a subject. The methods generally comprise the steps of assessing a biological sample harvested from the subject for the presence of a pentraxin-3 (PTX3) polypeptide, and correlating the presence of a PTX3 polypeptide to the risk for or the presence of asthma in the subject. Suitable biological samples are exemplified by serum, broncho-alveolar lavage fluid, and airway smooth muscle cells.

One exemplary embodiment relates to a method for assessing a biological sample harvested from a subject for the presence of a PTX3 polypeptide and correlating the results with the presence of a PTX3 polypeptide in corresponding samples from normal subjects lacking asthma. An elevated levels of a PTX3 polypeptide in the subject's biological sample relative to the presence of a PTX3 polypeptide in the normal subjects' samples is indicative of a risk for or a presence of asthma in the subject.

Another exemplary embodiment relates to a method for assessing a biological sample harvested from a subject for the presence of a PTX3 polypeptide and correlating the results with the presence of a PTX3 polypeptide in a plurality of biological samples harvested from the subject at successive time intervals from the subject. Significant variations in the presence of a PTX3 polypeptide in the biological sample compared to the presence of a PTX3 polypeptide in the plurality of biological samples harvested from the subject at successive time intervals from the subject, are indicative of a risk for or a presence of asthma in the subject.

Another exemplary embodiment relates to a method for assessing a first biological sample harvested from a subject for the presence of a PTX3 polypeptide at a time prior to treatment with an anti-asthma composition or an anti-inflammatory composition, assessing a second biological sample harvested from a subject for the presence of a PTX3 polypeptide at a time after treatment with the anti-asthma composition or the anti-inflammatory composition, and correlating the results. A significant drop in the presence of a PTX3 polypeptide in the post-treatment biological sample relative to the presence of a PTX3 polypeptide in the pre-treatment biological sample is indicative of a risk for or a presence of asthma in the subject.

Another exemplary embodiment relates to a method for culturing a first portion of a biological sample harvested from a subject with TNF-α and a second portion of the biological sample without TNFα. The two cultured portions are then assessed for the presence of a PTX3 polypeptide. A significant increase in the presence of a PTX3 polypeptide in the sample portion cultured with TNFα relative to the presence of a PTX3 polypeptide in the sample portion cultured without TNFα is indicative of a risk for or a presence of asthma in the subject.

### BRIEF DESCRIPTION OF THE DRAWINGS

These and other features of the invention will become more apparent from the following description in which reference is made to the appended drawings wherein:

FIG. 1(A) shows a protocol for sensitizing and challenging a group of mice to mimic acute asthma, and 1(B) shows a protocol for sensitizing and challenging a group of mice to mimic chronic asthma;

FIG. 2(A) is a chart comparing the levels of PTX3 polypeptides in serum from control mice, mice with acute asthma, and mice with chronic asthma, and 2(b) is a chart comparing the levels of PTX3 polypeptides in broncho-alveolar lavage from control mice, mice with acute asthma, and mice with chronic asthma;

FIG. 3 is a chart comparing the levels of PTX3 polypeptides in serum collected from healthy human volunteers, from non-asthmatic human volunteers with allergy symptoms, and from human volunteers with allergic asthma;

FIG. 4 is a chart showing Pentraxin 3 (PTX3) mRNA expressions in cultured human airway smooth muscle cells (HASMC), according to an exemplary embodiment of the invention. Data plotted are the mean±SD from three independent experiments (***, p<0.001);

FIGS. 5(A) and 5(B) show the time effects of TNF-α on PTX3 mRNA expression, according to an exemplary embodiment of the invention. Data represent the means±SD from 3 independent experiments;

FIG. 6 is a chart showing PTX3 expression in HASMCs from asthmatic patients compared with from normal donors, following stimulation with TNF-α;

FIG. 7 shows dose- and time-effects of TNF-α on PTX3 protein expression, according to an exemplary embodiment of the invention; (A) PTX3 release in the supernatant of growth-arrested HASMC stimulated for 24 h in the absence (medium) or presence of increasing concentrations (0.1, 1, 10 ng/ml) of TNF-α (*p<0.05, ***p<0.001, vs medium group); (B) PTX3 release in the supernatant of HASMCs stimulated for 6-72 hours with TNF-α (10 ng/ml). PTX3 was measured by ELISA;

FIGS. 8(A)-8(D) are micrographs showing detection of PTX3 polypeptides expressed in HASMC after in vitro stimulation by TNF-α. (A) cultured HASMC—negative control (primary antibody is rat IgG2b isotype-matched control antibody); (B) cultured HASMC from a normal subject; (C) HMASC from an asthmatic subject, and (D) HMASC from a normal subject stimulated with TNF-α (10 ng/ml); 400× magnification;

FIGS. 9(A)-9(B) are micrographs showing in vivo expression of PTX3 proteins in HASMC bundle within bronchial biopsies of a health control and an allergic asthmatic volunteer, respectively. 9(A) is a micrograph of a tissue section of a HASMC negative control sample (primary antibody was rat IgG2b isotype-matched control antibody); 9(B) is a micrograph of a tissue section of a HASMC from an-asthmatic subject; 9(C) and 9(D) are tissue samples of HASMC from asthmatic subjects stained with isotype control antibody; Magnification—100× for A, B, C and D; Immunochemistry was performed using lung biopsy sections from asthmatic volunteers and control volunteers in accordance with procedures approved by the

FIGS. 10(A)-10(C) are charts showing JNK inhibitor and p42/p44 ERK MAPK inhibitors abrogate TNF-α-mediated PTX3 release from HASMC: Growth-arrested cells were left unstimulated (medium alone) or treated with 10 ng/ml TNF-α 24 h with or without pretreatment for 1 h with inhibitors of: (A) JNK—SP500125, 50 nM, (b) p42/p44 ERK—U0126, 10 μM, AND (c) p38 MAPK—SB203580, 10 μM.

### DETAILED DESCRIPTION

The exemplary embodiments of the present invention relate to methods for a identifying a risk for or a presence of asthma in a subject. The methods generally comprise the steps of assessing a biological sample harvested from the subject for the presence of a pentraxin-3 (PTX3) polypeptide, and correlating the presence of PTX3 polypeptide to the risk for or the presence of asthma in the subject.

Pentraxins are a family of acute-phase proteins that are generally associated with inflammation responses. Their structures are characterized by multimeric, usually pentameric architectures and fall into two classes. Short pentraxins C-reactive protein (CRP) and serum amyloid P component (SAP) are produced by humans and mice, respectively, mainly by hepatocytes in the liver and by smooth muscle cells and endothelial cells in atheroscleric plaques. Long pentraxin 3 (PTX3) shares many similarities with the short pentraxins, but has an unrelated long N-terminal domain coupled to the C-terminal pentraxin domain. PTX3 also differs from the short pentraxins in its gene organization, cellular sources, inducting stimuli and ligands recognized.

PTX3 polypeptide is produced by several cell types in response to primary inflammatory signals. Known PTX3 polypeptide-producing cells include mononuclear phagocytes, myeloid dendritic cells, fibroblasts, epithelial cells, endothelial cells, embryonal carcinoma cells, and vascular smooth muscle cells. The primary inflammatory signals causing PTX3 production include lipopolysaccharide (LPS), cytokines such as tissue necrosis factor α (TNFα), interleulin-1β (IL-1β), and toll-like receptor (TLR) protein engagement of microbial cells. PTX3 binds to complement component CIq thereby activating the complement cascade system thereby enable the phagocytosis of apoptotic cells and microorganisms.

It has been surprisingly discovered that the presence of an elevated level of a PTX3 polypeptide and/or fragments and/or portions thereof in a biological sample taken from a subject compared to the level of or absence of PTX3 polypeptide and/or fragments and/or portions thereof in reference biological samples taken from healthy asthma-free subject can be correlated to the risk for or presence of asthma in the subject. Furthermore, changes in the presence of a PTX3 polypeptide and/or fragments and/or portions thereof in a plurality of biological samples obtained at successive time intervals from the subject can be correlated to the risk for or presence of asthma in the subject. Additionally, determining the presence of PTX3 polypeptide in a first biological sample collected from a subject at a time prior to treatment with an anti-asthma composition or an anti-inflammatory composition, then determining the presence of PTX3 polypeptide in a second biological sample collected at a time following treatment with the anti-asthma composition or the anti-inflammatory composition, and then correlating the data from the first biological sample and second biological sample, can enable determination of the risk for or presence of asthma in the subject. Furthermore, the correlation can enable determination of the subject's responsiveness to treatment with the anti-asthma composition or the anti-inflammatory composition. Alternatively, the risk for or presence of asthma in a subject can be determined by culturing a first portion of a biological sample with TNFα and culturing a second portion of the biological sample without TNFα, then determining the presence of PTX3 polypeptide and/or fragments and/or portions thereof in the first cultured portion of the biological sample and the second cultured portion of the biological sample. The data can be correlated to determine the risk for or presence of asthma in the subject.

Suitable biological samples are blood, serum, fluid from a from a bronchiolar lavage (BAL), and airway smooth muscle cells (ASMC). A non-liquid ASMC sample may be digested, extracted or otherwise rendered to a liquid form. Various methods for obtaining biological samples are known, and the suitability of a particular type of biological sample, and methods for obtaining it will be known to those skilled in the art. In some exemplary embodiments, the biological sample may comprise cells that may be cultured or grown to increase the quantity, for example, cells from a sample of BAL fluid. The cells once cultured and/or increased in quantity, may be subjected to further analysis for particular nucleic acid molecules, polypeptides or the like as described herein. A biological sample may comprise various polypeptides, or portions or fragments of polypeptides, including PTX3 or portions or fragments thereof. The body fluid may be employed in an undiluted from (e.g. “neat”) or may be prepared (concentrated or diluted) such that the level of PTX3 polypeptide or portion or fragment thereof, or a nucleic acid encoding a PTX3 polypeptide or portion or fragment thereof is at a level to fall within a linear portion of a standard curve. Preparation of a standard curve and identification of the linear portion is within the ability of one skilled in the art. One or more than one biological samples may be collected at any one time. A biological sample or samples may be taken from a subject at any time, including before initiation of a therapeutic regimen, during a therapeutic regimen, or at the conclusion of a therapeutic regimen.

The term “subject” or “patient” generally refers to mammals and other animals including humans and other primates such as chimpanzees or monkeys, companion animals, zoo, and farm animals, including, but not limited to, cats, dogs, rodents, rats, mice, hamsters, rabbits, horses, cows, sheep, pigs, goats, poultry, etc. A subject includes one who is to be tested, or has been tested for prediction, assessment or diagnosis of asthma, or for response to a therapeutic agent for use in the treatment of asthma. The subject may have been previously assessed or diagnosed with asthma using other methods (e.g. the subject has asthma), such as those described herein or those in current clinical practice, or may be selected as part of a general population (a control subject). A subject may be considered at risk of having, or suspected of having asthma, if the subject is a relative of a person diagnosed with asthma, or suspected of having asthma, or if the subject is exposed to an environment where factors may predispose the subject to asthma (e.g. the subject is a smoker, or is exposed to chemicals or allergens as a part of their lifestyle). A subject may be considered suspected of having asthma if the subject displays one or more of the clinical symptoms of asthma (e.g. wheezing, coughing, dyspnea, chest tightness or the like).

An exemplary embodiment of the present invention relates to a diagnostic method comprising testing for the presence, absence or amount of a PTX3 polypeptide molecule in a biological sample from a subject, and assessing the severity of asthma, based at least in part on the presence, absence or amount of the PTX3 polypeptide. An exemplary PTX3 polypeptide molecule is shown in SEQ ID NO: 1. An increase in the quantity of PTX3 polypeptide molecules, relative to a standard curve or control sample, or relative to the quantity of PTX3 polypeptide molecules in a previous sample obtained from the same subject, may be indicative of the severity of asthma increasing, and/or an increase in inflammation of the subject's airway. Similarly, no change (or no significant change) in the level of PTX3 polypeptide molecules (compared to a previous test) is indicative of the little to no change in the severity of asthma, and/or little to no change in inflammation of the subject's airway. A decrease in the quantity of PTX3 polypeptide molecules is indicative of the severity of the asthma decreasing, and/or a decrease in inflammation of the subject's airway.

A change in the levels of PTX3 polypeptides may also refer to a ratio, or a net value following subtraction of a baseline value. A change may also be represented as a ‘fold-change’, with or without an indicator of directionality (increase or decrease/up or down). The increase or decrease in expression of a marker may also be referred to as ‘down-regulation’ or ‘up-regulation’, or similar indicators of an increase or decrease. PTX3 polypeptide may be present in a first biological sample, and absent in a second biological sample; alternately the PTX3 polytpeptides may be present in both, with a statistically significant difference between the two. Expression of the presence, absence or relative levels of PTX3 polytpeptides in a biological sample may be dependent on the nature of the assay used to quantify or assess the marker, and the manner of such expression will be familiar to those skilled in the art.

A fold-change of PTX3 polytpeptides in a subject, relative to a control may be at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 or more, or any amount therebetween. The fold change may represent a decrease, or an increase, compared to the control value.

A fragment or portion of a polypeptide includes a peptide or polypeptide comprising a subset of the amino acid complement of a particular protein or polypeptide. The fragment can include an epitope for an antibody or antibodies used to specifically detect the polypeptide. The fragment may also comprise a region or domain common to proteins of the same general family, or the fragment may include sufficient amino acid sequence to specifically identify the full-length polypeptide from which it is derived.

A polypeptide, or fragment or portion of a polypeptide may range in size from as small as 4-6 amino acids to the “full-length” of the polypeptide. For example, a fragment or portion may be from about 1% to about 10%, from about 10% to about 20%, from about 20% to about 30%, from about 30% to about 40%, from about 40% to about 50%, from about 50% to about 60%, from about 60% to about 70%, from about 70% to about 80%, from about 80% to about 90% or from about 90% to about 100% of the full-length polypeptide. Alternately, a fragment or portion may be from about 4 to about 10, or any amount therebetween, from 10 to about 50, or any amount therebetween, from about 50 to about 100 or any amount therebetween, from about 100 to about 150, or any amount therebetween, from about 150 to about 250 or any amount therebetween, from about 250 to about 500 or any amount therebetween. Alternately, a fragment or portion may be 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acids long.

A polypeptide, or fragment or portion thereof is specifically identified when its sequence may be differentiated from others found in the same phylogenetic Species, Genus, Family or Order. Such differentiation may be identified by comparison of sequences. Comparisons of a sequence or sequences may be done using a BLAST algorithm (Altschul et al. 1009. J. Mol. Biol 215:403-410). A BLAST search allows for comparison of a query sequence with a specific sequence or group of sequences, or with a larger library or database (e.g. GenBank or GenPept) of sequences, and identify not only sequences that exhibit 100% identity, but also those with lesser degrees of identity.

Examples of assays and methods for detection of PTX3 polypeptides or fragments or portions thereof in a biological sample are known to those skilled in the art. Polypeptides or complexes comprising specific polypeptides, or fragments or portion thereof may be specifically identified and/or quantified by a variety of methods known in the art and may be used alone or in combination. Immunologic- or antibody-based techniques include enzyme-linked immunosorbent assay (ELISA), radioimmunoassay (RIA), western blotting, immunofluorescence, microarrays, antibody arrays, peptide arrays, some chromatographic techniques (i.e. immunoaffinity chromatography), flow cytometry, immunoprecipitation, microsphere-based multianalyte diagnostics and the like. Such methods are based on the specificity of an antibody or antibodies for a particular epitope or combination of epitopes associated with the protein or protein complex of interest. Non-immunologic methods include those based on physical characteristics of the protein or protein complex itself. Examples of such methods include electrophoresis, some chromatographic techniques (e.g. high performance liquid chromatography (HPLC), fast protein liquid chromatography (FPLC), affinity chromatography, ion exchange chromatography, size exclusion chromatography and the like), mass spectrometry, iTRAQ®, iCAT® or SELDI proteomic mass spectrometric based method, sequencing, protease digests, and the like (iTRAQ® is a registered trademark of Applera Corporation, 850 Lincoln Centre Drive Foster City Calif. 94404; iCAT® is a registered trademark of the University of Washington 4311 11th Avenue Northeast, Suite 500 Campus Box 354990 Seattle Wash. 981054608).

Such methods are based on the mass, charge, hydrophobicity or hydrophilicity, which is derived from the amino acid complement of the protein or protein complex, and the specific sequence of the amino acids. Examples of methods employing mass spectrometry include those described in, for example, PCT Publication WO 2004/019000, WO 2000/00208, U.S. Pat. No. 6,670,194. Immunologic and non-immunologic methods may be combined to identify or characterize a protein or protein complex. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like. An increase or decrease in PTX3 may be relative to a positive or negative control, or compared to a predetermined threshold.

Standard reference works setting forth the general principles of immunology, and various immunologically based detection methods known to those of skill in the art include, for example: Harlow and Lane, Antibodies: A Laboratory Manual, 2d Ed., Cold Spring Harbor Laboratory Press, Cold Spring Harbor, N.Y. (1999); Harlow and Lane, Using Antibodies: A Laboratory Manual. Cold Spring Harbor Laboratory Press, New York; Coligan et al. eds. Current Protocols in Immunology, John Wiley & Sons, New York, N.Y. (1992-2006); and Roitt et al., Immunology, 3d Ed., Mosby-Year Book Europe Limited, London (1993).

An “antibody”, as used herein, includes polyclonal antibodies from any native source, and native or recombinant monoclonal antibodies of classes IgG, IgM, IgA, IgD, and IgE, hybrid derivatives, humanized or chimeric antibodies, and fragments of antibodies including Fab, Fab′, and F(ab′)2, and the products of a Fab or other immunoglobulin expression library. The antibody may be naturally-occurring, e.g., isolated and/or purified from an animal (e.g., mouse, rabbit, goat, horse, chicken, hamster, human, or the like). The antibody can be in monomeric or polymeric form. The antibody, or antigen binding portion thereof, can be modified to comprise a detectable label, such as, for instance, biotin, a radioisotope, a fluorophore (e.g., fluorescein isothiocyanate (FITC), phycoerythrin (PE), Alexa488 or other Alexa dyes), an enzyme (e.g., alkaline phosphatase, horseradish peroxidase), or particles of an element (e.g., gold particles).

Antibodies may be made according to any of several methods known in the art. In some exemplary embodiments, commercially available antibodies may be employed in the methods of the present invention. The antibodies may be unlabelled and used in combination with a secondary, labeled detection antibody, or a detection label may be conjugated to the anti-PTX3 antibody. Examples of such commercially available anti-PTX3 antibodies include polyclonal or monoclonal antibodies to mouse and/or human PTX3 obtained from R&D Systems® (e.g. catalog numbers AF1826, PP-PPJ0069-00, 2ZPPJ0069H, AF2558, MAB2166, MAB1826, MAB21661, AF2166, BAF2166, BAF1826; R&D Systems® is a registered trademark of Techne Corporation 614 McKinley Place N.E. Minneapolis Minn. 55413). Other antibodies, and suppliers that provide them, that may be used with the exemplary methods described herein will be known to those skilled in the art.

A hybridoma method may be used to make monoclonal antibodies (Kohler et al. (1975) Nature 256:495). Alternately, monoclonal antibodies may be made by recombinant DNA methods (for example U.S. Pat. No. 4,816,567). Monoclonal antibodies may also be isolated from a phage antibody library, for example, by using the techniques described in Clackson et al. (1991) Nature 352:624-628; and Marits et al. 1991 J. Mol. Biol. 222:581-597. Methods of making and characterizing chimeric or humanized antibodies are known in the art, and are described in, for example, Kashmiri et al., 2005. Methods 36:25-34; Gonzales et al., 2005. Tumor biology 26:31-43.

As an alternate to an antibody that binds PTX3, it will be apparent to one skilled in the art that any agent having affinity and binding selectively to PTX3 may be useful for assaying for PTX3. In some exemplary embodiments, the compound may be a peptidomimetic, or an aptamer. A peptidomimetic is a synthetic structure that may, or may not contain amino acids and/or peptide bonds, but retains the structural and functional feature of a PTX3 polypeptide-binding reagent, such as an antibody. An aptamer refers to a short oligonucleotide that can bind an antigen (e.g. PTX3 polypeptide); an aptamer may be at least 10, 20, 30, 40, 50, 60, 70 or more bases, or base pairs in length, or any amount therebetween.

An exemplary embodiment of the present invention relates to a method of identifying a subject having, at risk of having, or suspected of having asthma, the method comprising obtaining a biological sample from the subject and testing for the presence, absence or amount of a nucleic acid molecule encoding a PTX3 polypeptide, or a fragment or portion of the nucleic acid encoding a PTX3 polytpeptides in the biological sample.

For example, detection or determination, and in some cases quantification, of a nucleic acid may be accomplished by any one of a number methods or assays employing recombinant DNA technologies known in the art, including but not limited to, as sequence-specific hybridization, polymerase chain reaction (PCR), RT-PCR, microarrays and the like. Such assays may include sequence-specific hybridization, primer extension, or invasive cleavage. Furthermore, there are numerous methods for detecting, analyzing or detecting and analyzing the products of each type of reaction (for example, fluorescence, luminescence, mass measurement, electrophoresis, etc.). Furthermore, reactions can occur in solution or on a solid support such as a glass slide, a chip, a bead, or the like.

Methods of designing and selecting probes for use in microarrays or biochips, or for selecting or designing primers for use in PCR-based assays are known in the art. Once the marker or markers are identified and the sequence of the nucleic acid determined by, for example, querying a database comprising such sequences, or by having an appropriate sequence provided (for example, a sequence listing as provided herein), one of skill in the art will be able to use such information to select appropriate probes or primers and perform the selected assay.

Standard reference works setting forth the general principles of recombinant DNA technology known to those of skill in the art include, for example: Ausubel et al. (Current Protocols In Molecular Biology, John Wiley & Sons, New York, 1998 and Supplements to 2001); Sambrook et al, Molecular Cloning: A Laboratory Manual (2d Ed., Cold Spring Harbor Laboratory Press, Plainview, N.Y., 1989); Kaufman et al, Eds. (Handbook Of Molecular And Cellular Methods In Biology And Medicine, CRC Press, Boca Raton, 1995); McPherson, Ed. (Directed Mutagenesis: A Practical Approach, IRL Press, Oxford, 1991).

Therapeutic regimens for asthma are known in the art. Once a subject is diagnosed as having asthma, the subject may be provided a therapeutic regimen. A therapeutic regimen may be selected to, for example, minimize airway impairment, minimize acute and/or chronic symptoms or achieve and/or maintain baseline (normal) pulmonary function, or a combination thereof. A therapeutic regimen for asthma may include one or more of control or reduction of triggering factors, drug therapy, and/or regular monitoring of the severity of the asthma and/or the inflammatory state of the airway.

Control of triggering factors may include lifestyle changes (use of low-allergen linens, bedding and the like; changes in domestic hygiene; diet changes, etc). A variety of drug classes may be used in the treatment of asthma. Examples of drug classes include bronchodilators (e.g. beta-2 agonists, anticholinergics), corticosteroids, leukotriene modifiers, mast cell stabilizers and methylxanthines and the like. Suitability of a particular drug, dosage and mode of administration is known to those skilled in the art, and is discussed in, for example, chapter 27 “Pharmacotherapy of Asthma”, Goodman & Gilman's The Pharmacological Basis of Therapeutics 11 th edition. 2006. L L Brunton, editor. McGraw-Hill, New York.

Another exemplary embodiment of the invention relates to a method of assessing the effectiveness of a therapeutic regimen for treatment of asthma comprising: testing for the presence, absence or amount of a PTX3 polypeptide, in a biological sample of a subject undergoing the therapeutic regimen; and, assessing the effectiveness of the therapeutic regimen based at least in part on the presence, absence or amount of the PTX3 polypeptide.

An increase in PTX3 polypeptide, relative to a standard curve or control sample, or relative to a previous sample taken from the same subject, is indicative of the therapeutic regimen having little, or no effect on the subject's asthma; or little, or no reduction in the inflammation of the subject's airway (e.g. the subject may have an increase in the PASS or PRAM score, or a change in the NHLBI category that increases in severity). Similarly, no change in the level of PTX3 polytpeptides or a fragment or portion thereof (compared to a prior testing) may be indicative of the therapeutic regimen having little or no effect (e.g. no change in the PASS or PRAM score, or no change in the NHLBI category). A decrease in PTX3 polytpeptides is indicative of the therapeutic regimen reducing the severity of the asthma, and/or a reduction in the inflammation of the subject's airway (e.g. a decrease in the PASS or PRAM score, or a change in the NHLBI category that decreases in severity).

The present invention will be further illustrated in the following examples. However it is to be understood that these examples are for illustrative purposes only, and should not be used to limit the scope of the present invention in any manner.

### EXAMPLES

### Example 1

### Detection and Assessment of PTR3 in Asthmatic Mice

Animals: Female BALB/c mice (7 weeks old) were obtained from the Central Animal Care Services, University of Manitoba (Winnipeg, MB, CA). The experiments were approved by the Animal Care and Use Committee at the University of Manitoba, and the investigators adhered to Canadian Council on Animal Care (CCAC) guidelines for humane treatment of animals. Protocol for sensitization and challenge

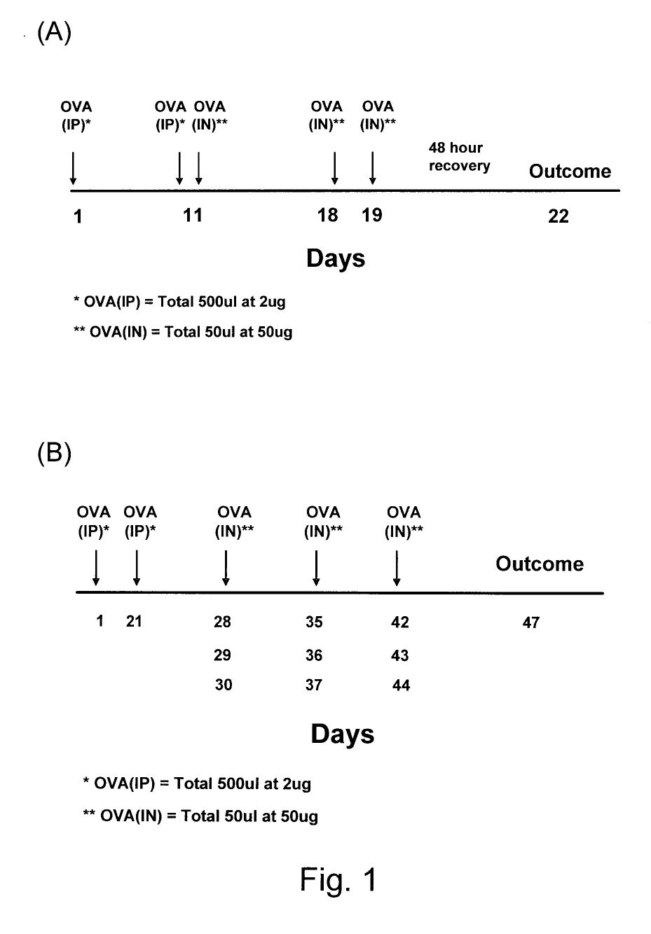

Mice were divided into three groups (three mice per groups 1 and 2; six mice in group 3). Group 1 was the “acute” group. The three mice in Group 1 were sensitized twice (at day 1 and 11) by intraperitoneal injections of 2 μg of ovalbumin (OVA) (Sigma-Aldrich, grade IV) in a volume of 500 μl PBS (FIG. 1(A)). These mice were challenged three times (at days 11, 18 and day 19) by an application of an intranasal droplet of 50 μg OVA in a volume of 50 μl. The Group 1 mice were harvested 3 days after the third challenge i.e., on day 22 (FIG. 1 (A)).

Group 2 was the “chronic” group. The three mice in Group 2 were sensitized twice (at day 1 and 21) by intraperitoneal injections of 2 μg of OVA in a volume of 500 μl PBS (FIG. 1(B)). These mice were then challenged nine times (at days 28, 29, 30, 35, 36, 37, 42, 43, 44) by application of an intranasal droplet of 50 μg OVA in a volume of 50 μl (FIG. 1(B)). The Group 1 mice were harvested 3 days after the third challenge, i.e., on day 47 (FIG. 1 (B)). Their serum samples and BAL samples were collected using standard protocols and assayed for the presence of PTX3 using and ELISA test.

Group 3 was the “control” group. These mice were injected twice (at day 1 and 11) by intraperitoneal injections 500 μl of sterile PBS. Three control mice were harvested on day 22 at the same time that the Group 1 mice were harvested. Serum samples and BAL samples were collected from each of the mice in Groups 1 and 3, using standard protocols as for example disclosed by Gounni et al (2001, Molec. Med, 7:344-354). Each serum and BAL sample was then assayed for the presence of PTX3. The remaining three control mice were harvested on day 47 at the same time that the Group 2 mice were harvested. Each serum and BAL sample was then assayed for the presence of PTX3.

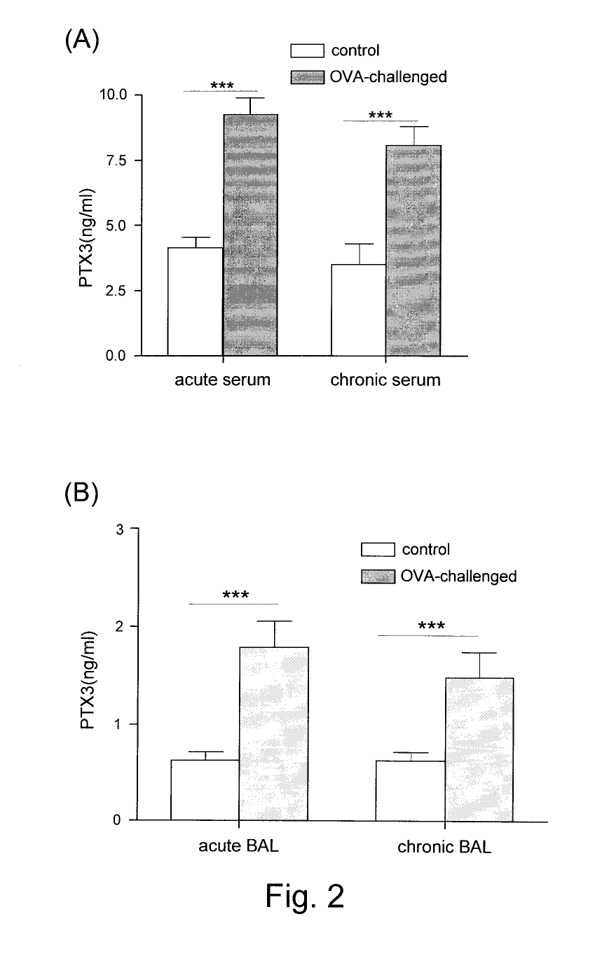

The PTX3 data generated from the serum samples and BAL samples from each group of animals were statistically analysed using GraphPad Prism Software Version 3.02 for Windows (GraphPad Software, San Diego, Calif., USA). Comparison between expression levels of PTX-3 in the subgroups were studied using ANOVA with Bonnferroni post test comparison. The results are shown in FIGS. 2(a) and 2(B). PTX3 plasma levels were significantly higher in OVA-challenged groups compared with the control group in both the “acute” protocol and the “chronic” protocol (FIGS. 2(a) and 2(B)).

### Example 2

### Detection and Assessment of PTR3 in Serum Samples from Human Subjects

Serum samples were collected from three groups of human volunteers. The first group comprised normal healthy donors. The second group comprised non-asthmatics that were exhibiting allergic reactions. The third group comprised individuals that were diagnosed as allergic asthmatics. This study was approved by the Ethics Committee of the Faculty of Medicine, University of Manitoba, Winnipeg, Canada and written informed consent was obtained from each participant. In response to advertisements, individuals 18-45 years old were recruited in each of three groups: allergic individuals with mild asthma, allergic non-asthmatics, and healthy donors. The clinical diagnosis of allergic asthma was determined by: (i) history of asthma symptoms (wheeze, cough, and/or shortness of breath) during the short (6-8 week long) local grass pollen season, controlled with albuterol as needed; (ii) positive epicutaneous test to mixed grass pollen (wheal diameter at least 3 mm more than histamine control wheal) to an epicutaneous test with mixed grass pollen; (iii) 15% or greater improvement in forced expiratory volume in one second (FEV₁) after inhalation of albuterol (200 μg) from a metered-dose inhaler. The clinical designation of allergic non-asthmatic was determined by: (i) history of allergic rhinitis symptoms (sneezing, nasal itching, discharge, and/or congestion) during the short local grass pollen season, relieved by an H₁-antihistamine as needed; (ii) positive epicutaneous test to mixed grass pollen (wheal diameter at least 3 mm more than histamine control wheal), (iii) no history of asthma symptoms at any time of year, normal FEV₁ and no change in FEV₁ after albuterol 200 μg from a metered-dose inhaler. The healthy donors had no history of asthma, allergic rhinitis, or other allergic disease, negative epicutaneous tests to mixed grass pollen, normal FEV₁ and no change in FEV₁ after albuterol 200 μg by metered-dose inhaler. Study participants had not received allergen-specific immunotherapy. For three days before collection of a 40 ml blood sample, all participants refrained from using all medications, including β₂-adrenergic agonists and H₁-antihistamines. Participants who reported an upper respiratory tract infection within the previous month were excluded from the study.

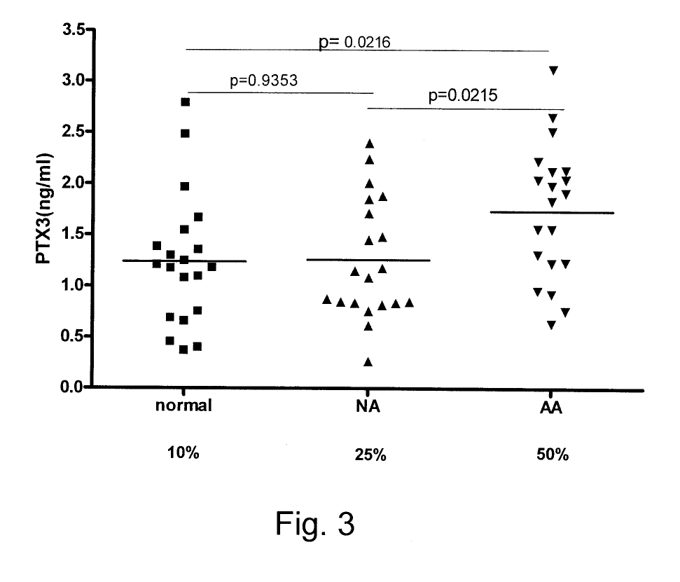

The levels of PTX3 in the serum samples were determined by the following process using an ELISA test. The PTX3 data generated from the serum samples from each group of volunteers were statistically analysed using the ANOVA test combined with a post-hoc Bonferroni analysis using GraphPad Prism Software Version 3.02 for Windows (GraphPad Software, San Diego, Calif., USA). Non-parametric data were analyzed using the Kruskal-Wallis test followed by the Mann-Whitney U-test. P values were considered significant at 0.05 levels. The results are shown in FIG. 3. Allergic asthmatics (AA) displayed statistically higher levels (P<0.05) of PTX3 level compared to allergic non-asthmatics (NA) or healthy donors (normal). Furthermore, in term of frequency, 50% of allergic asthmatics displayed higher level of PTX3 compared to 25% and 15% in allergic non-asthmatics and healthy subjects respectively (FIG. 3).

### Example 3

### Detection and Assessment of PTR3 in Human Airway Smooth Muscle Cell Samples from Human Subjects

Reagents and Antibodies.

Recombinant human TNF-α, mouse anti-human pentraxin-3 (PTX3) antibody (Ab), biotinylated goat anti-human PTX3, and recombinant human PTX3 were purchased from R&D Systems®. Monoclonal antibody to PTX3 (MNB1) was purchased from ALEXIS Biochemicals. Rat IgG_{2b} control were from Sigma-Aldrich® (Oakville, Ontario, Canada). Goat anti-rat IgG F(ab′)2 Alexa Fluor® 488 and ProLong® anti-fade were obtained from Molecular Probes® (Eugene, Oreg.). Goat serum and normal human serum were from Cedarlane (Toronto, Ontario, Canada). FBS was from HyClone® (Logan, Utah). DMEM, Ham's F-12, trypsin-EDTA, antibiotics (penicillin, streptomycin), dNTP, SuperScript® reverse transcriptase, and Taq polymerase were from Invitrogen® Life Technologies (Grand Island, N.Y.). PI (Propidium iodide) was from Sigma-Aldrich. The p38 MAPK inhibitor, SB-203580 [4-(4-fluorophenyl)-2-(4-methyl-sulfinylphenyl)-5-(4′-pyridyl)-1H-imidazole], the p42/p44 ERK inhibitor, U-0126 [1,4-diamino-2,3-dicyano-1,4-bis(2-aminophenylthio)butadiene], and the JNK inhibitor, SP600125, were purchased from Calbiochem® (Mississauga, Ontario, Canada). Unless stated otherwise, all other reagents were obtained from Sigma-Aldrich®. (Alexa Fluor, ProLong, Molecular Probes, are registered trademarks of Molecular Probes, Inc., Eugene Oreg., USA) (HyClone is a registered trademark of Hyclone Laboratories Inc., Logan Utah. USA) (SuperScript, Invitrogen are registered trademarks of Invitrogen Corporation, Carlsbad Calif., USA) (Calbiochem is a registered trademark of EMD Chemicals Inc., Gibbstown, N.J., USA) (Sigma-Aldrich is a registered trademark of Sigma-Aldrich Biotechnology Holding Company Inc., Saint Louis Mo., USA).

Preparation of HASMC.

Bronchial airway smooth muscle cells from human subjects (HASMC) were obtained from macroscopically healthy segments of the main bronchus after lung resection from surgical patients and asthmatic patients in accordance with procedures approved by the Human Research Ethics Board of the University of Manitoba, Winnipeg, Manitoba, Canada. Briefly, the muscle layer from each bronchial segment was dissected free from adventitia and submucosa under a binocular dissection microscope and then minced, and cells were dissociated enzymatically (600 U/ml collagenase I, 10 U/ml elastase, 2 U/ml Nagarse protease) for up to 60 min. Cells were seeded at a density of 8,000 cells per cm2 and grown at 37° C. in DMEM supplemented with 10% FBS, sodium pyruvate (1 mM), L-glutamine (2 mM), nonessential amino acid mixture (1:100), gentamicin A (50 μg/ml), and amphotericin B (1.5 μg/ml). Media were replaced every 2 days, and confluent cultures were passaged and reseeded using a split ratio of 1:4. At confluence, primary HASMC exhibited spindle morphology and a hill-and-valley pattern that is characteristic of smooth muscle in culture. Moreover, using cultures up to passage 5, over 90% of the cells at confluence retain smooth muscle-specific actin, SM22, and calponin protein expression and mobilize intracellular Ca²⁺ in response to acetylcholine, a physiologically relevant contractile agonist (Hirst, S. J. 2003. Respir. Physiol. Neurobiol. 137:309-326). The growth rates of the HASMC from all lung resection donors were similar to what has been reported previously for HASMC cultures from healthy human transplant donors. The clinical characteristics of the subjects shown in Table 1. In all experiments, cells were used at passages 2-5.

TABLE 1
Clinical characteristics of the subjects*

| | Asthmatic patients | | Normal control subjects | |
|---|---|---|---|---|
| Number | 8 | | 5 | |
| Age, yrs (range) | 24.5 | (19-35) | 24.2 | (20-31) |
| Sex, Male/Female | 2/6 | | 2/3 | |
| Smoking history, yes/no | 2/6 | | 1/4 | |
| Atopy, yes/no | 8/0 | | 0/5 | |
| FEV1** (L) | 3.098 | (2.12-4.24) | 3.9 | (3.09-5.17) |
| FEV1 (%) | 87.4 | (69-122) | 99.6 | (86-108) |

*Data are expressed as medians with ranges in parentheses.
**FEV1—forced expiratory volume in 1 sec.

Cell Stimulation.

Confluent HASMC (passages 2-5) were growth-arrested by FBS deprivation for 48 h in Ham's F-12 medium containing 5 μg/ml human recombinant insulin, 5 μg/ml human transferrin, 5 ng/ml selenium, and antibiotics (100 U/ml penicillin and 100 μg/ml streptomycin). Cells were then stimulated in fresh FBS-free medium containing graded concentration (0.1, 1, 10, and 100 ng/ml) of human TNF-α, IL-4, PTX3 or medium alone. In some experiments, cells were pretreated for 1 h with U-0126 (10 μM), SB-203580 (10 μM), or SP600125 (50 nM) before stimulation for 24 h with TNF-α (10 ng/ml), at 10 ng/ml. Supernatants were collected at 24 and 48 h, centrifuged at 1,200 rpm for 7 min at 4° C. to remove cellular debris, and stored at −80° C. until analysis by ELISA.

ELISA Analysis of PTX3 Protein Release in Cell Supernatants.

Immunoreactive PTX3 within the supernatants was quantified using ELISA with matched antibodies according to basic laboratory protocols provided by the manufacturer (R&D Systems Inc., Minneapolis, Minn.). PTX3 protein was quantified in reference to serial dilutions of recombinant standards falling within the linear part of the standard curve for each specific PTX3 measured. Each data point represents readings from a minimum of four independent assays wherein each assay was performed in duplicate.

RNA Isolation and RT-PCR.

Confluent HASMC (passages 2-5) were growth-arrested for 48 h in serum-free medium as described above. Cells were then stimulated in fresh FBS-free medium containing human recombinant TNF-α (10 ng/ml), IL-4, or medium alone for 2, 6, and 24 h. Cells were harvested, and total cellular RNA was extracted using TRIzol® method (Invitrogen Life Technologies, Gaithersburg, Md.). The RNA concentration and purity were assessed with optical density measurements. Reverse transcription was performed by using 2 μg of total RNA in a first-strand cDNA synthesis reaction with SuperScript® reverse transcriptase as recommended by the supplier (Invitrogen Life Technologies). PCR was performed by adding 2 μl of the reverse transcription product into 25 μl of total volume reaction containing 1× buffer, 200 μmol of each dNTP, 20 pmol of each oligonucleotide primer, and 0.2 unit of AmpliTaq® polymerase. Oligonucleotide primers of the human PTX3 were synthesized as follows: The sequences of primers were as follows: PTX3 forward primer, 5′-GGGACAAGCTCTTCATCATGCT-3′ (SEQ ID NO: 2); reverse primer, 5′-GTCGTCCGTGGCTTGCA-3′ (SEQ ID NO: 3); primers for housekeeping gene glyceraldhyde-3-phosphate dehydrogenase (GAPDH) is forward primer 5′-AGCAATGCCTCCTGCACCACCAAC-3′ (SEQ ID NO: 4) and reverse primer 5′-CCGGAGGGGCCATCCACAGTCT-3′ (SEQ ID NO: 5). PCR (PTX3, 35 cycles; GAPDH, 25 cycles) was conducted in a thermal cycler (Mastercycler®, Eppendorf®). Each cycle included denaturation (94° C., 1 min), annealing (PTX3, 62° C., 1 min; GAPDH, 55° C., 1 min), and extension (72° C., 1 min 30 s). The initial denaturation period was 5 min, and the final extension was 10 min. The size of the amplified PTX3 fragment is 97 bp, while the size of the GAPDH fragment is 137 bp. GAPDH was amplified as internal control. Amplified products were analyzed by DNA gel electrophoresis in 2% agarose and visualized by ethidium bromide staining under ultraviolet illumination. The specificity of the amplified band was confirmed by sequencing (data not shown). The PTX3 level was quantified by scanning densitometry and corrected for GAPDH in the same sample. (Trizol® is a registered trademark of Molecular Research Center Inc., Cincinnati Ohio, USA) (Mastercycler and Eppendorf are registered trademarks of Eppendorf AG, Hamburg Fed Rep Germany).

Quantitative Real-Time RT-PCR Analysis.

Total cellular RNA extraction and reverse transcription was performed as described above. PCR products were isolated from 2% wt/vol agarose gel using QIAEX® II Agarose Gel Extraction kit (Qiagen). The amount of extracted DNA was quantified by spectrophotometry and expressed as copy number. A serial dilution was used to generate each standard curve. For real-time quantitative PCR, each reaction contained the following: 1× LightCycler® DNA Master SYBR Green I (Roche), 25 mM MgCl₂, 0.5 μM each primer, 0.07 μM TaqStart® Ab (Clontech), and 10 μl (1:10 for PTX3 and 1:80 for GAPDH) of cDNA, in a final volume of 25 μl. After 10 min of denaturation at 95° C., the reactions were cycled 40 times for 5 s at 95° C., 10 s at the annealing temperature, and 7 s at 72° C. for GAPDH and 35 times for 10 s at 95° C., 10 s at the annealing temperature, and 32 s at 62° C. for PTX3. Product specificity was determined by melting curve analysis and by visualization of PCR products on agarose gels. Calculation of the relative amount of each cDNA species was performed according to standard protocols. Briefly, the amplification of PTX3 gene in stimulated cells was calculated first as the copy number ratio of PTX3 per copy of GAPDH and then expressed as normalized values of fold increase over the value obtained with unstimulated control cells. (QIAEX and Qiagen are registered trademarks of Qiagen GmbH Qiagen Str., Hilden, Fed Rep. Germany) (LightCycler and Roche are registered trademarks of Roche Diagnostics GMBH Ltd., Mannheim, Fed Rep. Germany) (SYBR is a registered trademark of Molecular Probes, Inc, Eugene, Oreg., USA) (TaqStart and Clontech are registered trademarks of Clontech Laboratories, Inc., Mountain View, Calif., USA).

Immunofluorescence.

Serum-fed HASMC were grown on eight-well glass slides (Nalge® Nunc®, Naperville, Ill.) (Nalge and Nunc are registered trademarks of Nalge NUNC International Corp., Rochester, N.Y., USA) and cultured up to semiconfluence. Slides were fixed with 4% paraformaldehyde, air-dried, and stored at −20° C. until use. Briefly, after treatment with a universal blocking solution for 30 min (Dakocytomation, Carpinteria, Calif., USA) (Dakocytomation® is a registered trademark of DakoCytomation Denmark AIS, Glostrup, DK), the slides were incubated with purified rat anti-human PTX3 Ab or matched control immunoglobulin at a final dilution of 10 μg/ml overnight at 4° C. and washed twice with Tris buffered saline (TBS). The slides were then incubated for 2 h at room temperature with donkey anti-rat IgG F(ab′)₂ Alexa Fluor® 488 (1:100 dilution) (Alexa Fluor is a registered trademark of Molecular Probes Inc., Eugene Oreg., USA). Slides were extensively washed with TBS and counterstained with the nuclear stain propidium iodide (PI) for 10 min (Sigma). After washing with TBS, the slides were mounted with ProLong® antifade (ProLong is a registered trademark of Molecular Probes Inc., Eugene Oreg., USA). Samples were photographed on Olympus AX-70 microscope with a Photometrics® PXL cooled CCD camera and Image-Pro® Plus software (Carsen Group) (Photometrics is a registered trademark of Roper Scientific Inc., Tucson Ariz., USA) (ImagePro is a registered trademark of Media Cybernetics LP, Silver Spring, Md., USA).

Immunohistochemistry.

Immunohistochemistry was performed using tissue sections prepared from segments of the main bronchus after lung resection from surgical patients. Deparaffinized sections were rehydrated in a series of graded concentrations of alcohol to water and then antigen retrieval by using microwave in citrate buffer. This was followed by incubation of the sections for 10 min in 0.25% Triton® X-100 in PBS at room temperature, followed by incubation with blocking solution (10% human normal serum, 10% goat serum in TBS) for 30 min at room temperature. Rat anti-human PTX3 monoclonal antibody or control IgG_{2b} (both at 2.5 μg/ml) were added, and sections were incubated overnight at 4° C. followed by biotinylated anti-rat IgG (H+L) 1:200 dilution incubated 1 hr at room temperature. Slides were then processed with streptavidin-alkaline phosphatase and developed by fast red staining. (Triton is a registered trademark of Union Carbide Corp., Midland, Mich., USA).

Statistical Analysis.

Data were obtained from experiments performed in triplicate and repeated at least three times, and results are expressed as means±SD. Statistical significance was determined using T test, and P values<0.05 were considered statistically significant.

### Example 4

### TNF-α Induces PTX3 mRNA Expression in HASMC

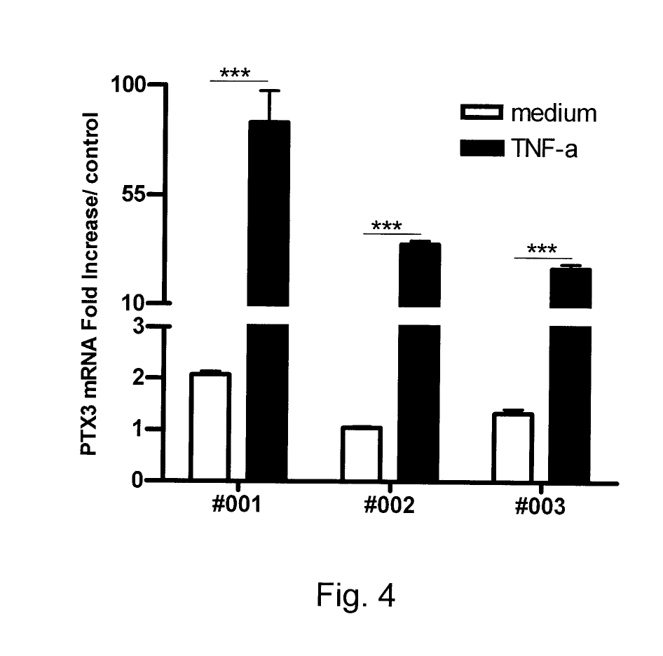

The effects of TNFα challenge on PTX3 expression in HASMC were assessed. Human smooth muscle cells were treated with medium alone, or medium containing TNF-α (10 ng/mL). After 6 hours exposure, RNA was isolated and RT-PCR performed as described. PTX3 mRNA was expressed as a ratio relative to a housekeeping gene, glyceraldehydes-3-phosphate dehydrogenase (GAPDH). Variability among primary cell lines was observed, however the overall trend was the same for each of the three cell lines (FIG. 4). HASMC demonstrate constitutive PTX3 mRNA expression that is enhanced significantly with TNF-α stimulation for 6 h-TNF-α induced a 30.93±11.25-fold increase of PTX3 mRNA, compared with unstimulated cells (medium-treatment only). IL-4 had no significant effect (data not shown).

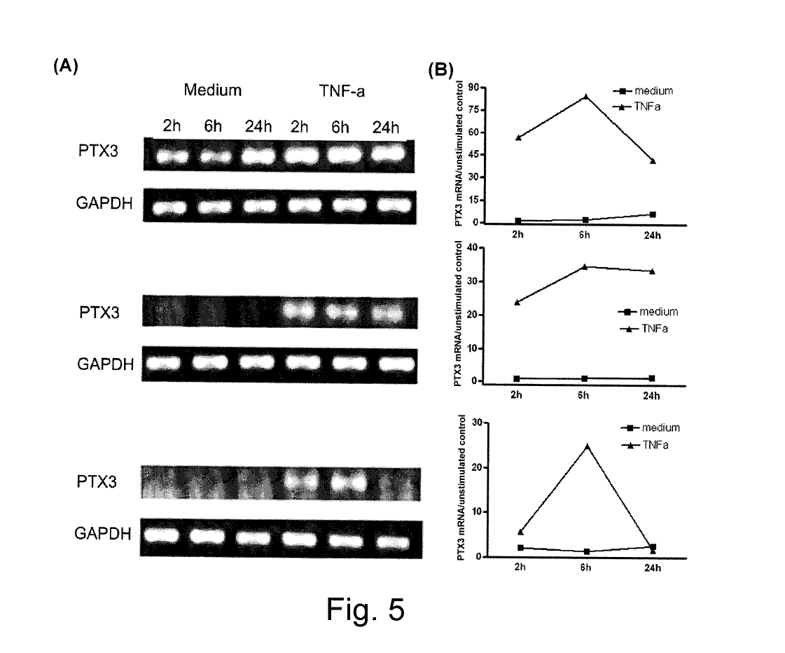

The effects of stimulation with TNF-α (10 ng/ml) for 2, 6, and 24 h on PTX3 mRNA expression in HAMSC were further assessed. (A) growth-arrested HASMC were left unstimulated (medium alone) or stimulated with TNF-α (10 ng/ml) for 2, 6, and 20 h. mRNA was analyzed by RT-PCR as described. GAPDH was again used as a reference and internal control. FIGS. 5A and 5B show data representative of 3 separate experiments. (B) PTX3 mRNA expression in TNF-α stimulated HASMC was analyzed by real-time RT-PCR as described. PTX3 mRNA was detectable after 2 hr stimulation with TNF-α, with a peak response at 6 h, which was reduced at 24 h. GAPDH products were of similar intensity between all samples, suggesting equality of the RNA preparations (FIGS. 5A and 5B).

### Example 5

### TNF-α Induces PTX3 Protein Release from HASMC

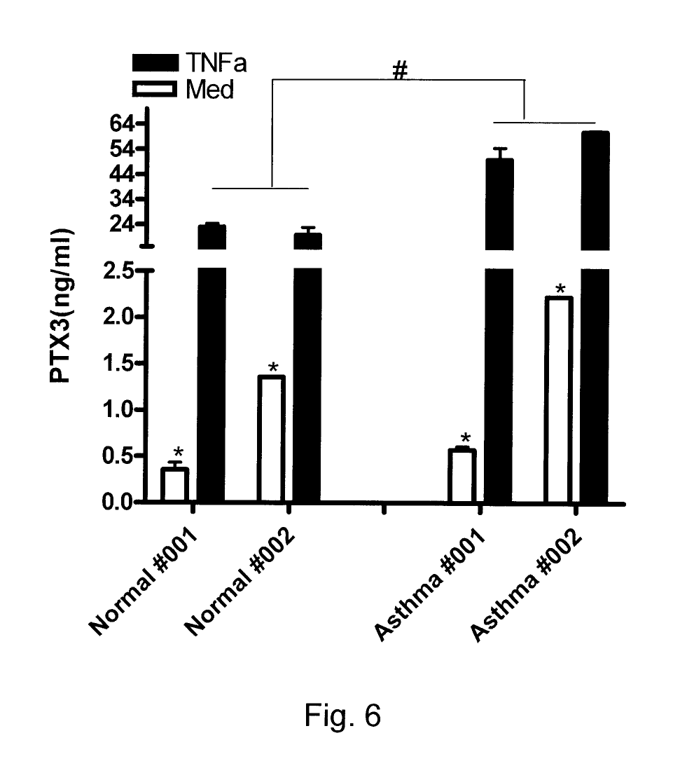

Assessments were made to determine if HASMC released PTX3 protein upon TNF-α stimulation. HASMCs were stimulated with TNF-α (10 ng/ml). Supernatants were harvested after 24 h and were tested for PTX3 by ELISA (*p<0.001, TNF-α-stimulated group vs medium group; #p<0.001, patients vs normal donors). HAMSC cells from two normal subjects and two asthmatic subjects were stimulated with TNF-α, and the cell culture supernatant assayed by ELISA to quantify PTX3 polypeptide. HASMC from asthma subjects demonstrated a significant increase in PTX3 polypeptide expression, compared to that of HASMC from normal subjects (FIG. 6).

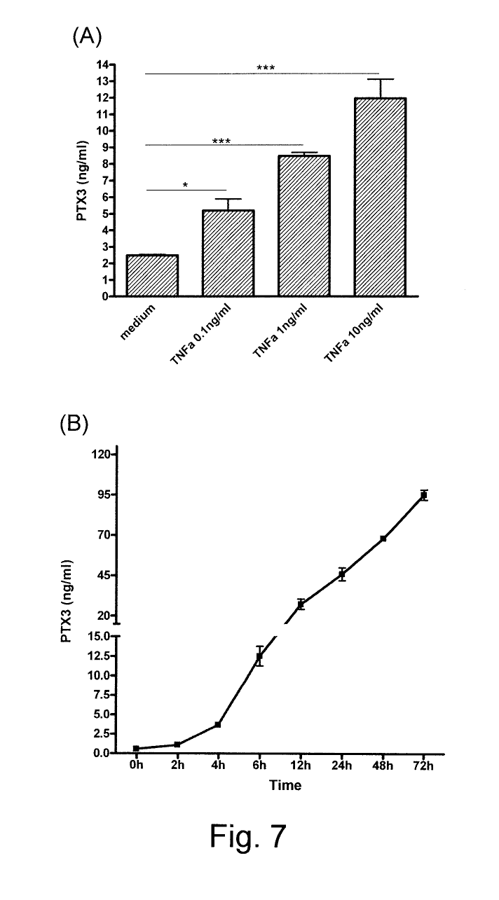

Assessments were made of the effects of dosage and exposure time of TNF-α on HASMC. Growth-arrested (serum-deprived) HASMC were stimulated for 24 h in the absence (medium) or presence of increasing concentrations (0.1, 1, 10 ng/ml) of TNF-α (FIG. 4A); Growth-arrested HASMC were incubated for various time-periods in the presence of TNF-α (10 ng/ml) (FIG. 7B). For both experiments, HASMC were stimulated with 0.1, 1, 10 or 100 ng/ml TNF-α, or medium alone for 24 h after which, PTX3 was measured by ELISA. Stimulation with TNF-α induced the release of PTX3 in a dose-dependent manner at 24 h, a statistically significant increase in PTX3 release from HASMC occurred with 0.1, 1, and 10 ng/ml TNF-α (*P<0.01 and***P<0.001), respectively (FIG. 7A) No significant PTX3 release could be detected in IL-4 (P>0.05, data not shown).

### Example 6

### Expression of PTX3 in Cultured Primary HASMC

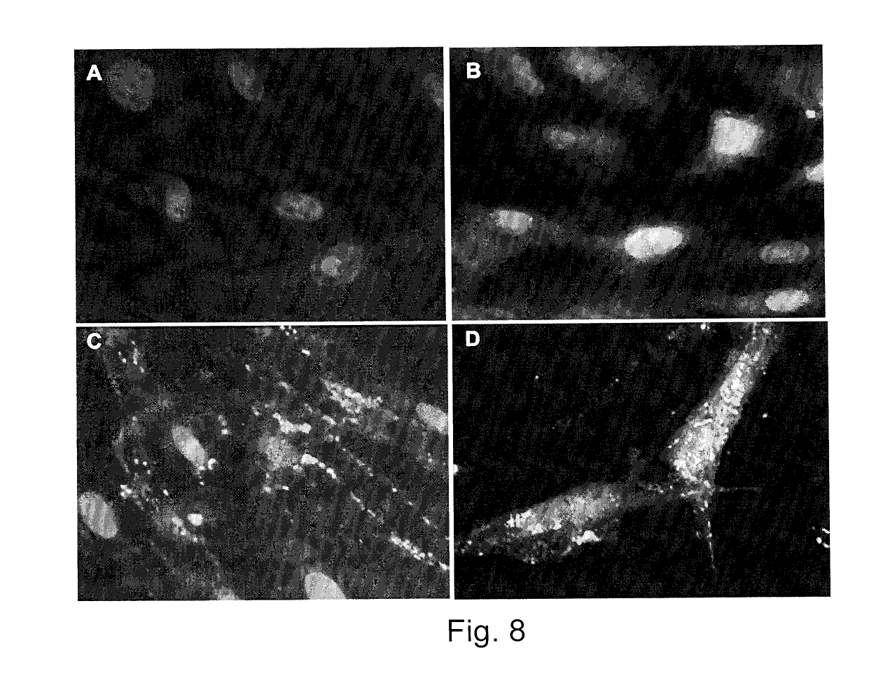

To further investigate the protein expression of PTX3 in HASMC, the HASMC were subjected to immunofluorescence staining. Staining was performed using rat anti-human PTX3 monoclonal antibody, and visualized with goat anti-rat IgG (ab′)₂—Alexa Fluor 488 labeled polyclonal antibody. Nuclei were counterstained with propidium iodide. Specific fluorescent staining was detected using the monoclonal antibody to PTX3 (MNB1) in cultured HASMC from normal subject (B), asthmatic patient (C) and from normal subject stimulated with TNF-α (10 ng/ml)(D), but not with rat IgG_{2b} isotype matched control (A). Cytoplasmic staining was clearly observed in normal HASMC (FIG. 8B); with an increase in punctate staining in HASMCs from asthmatic patients (FIG. 8C); and a further increase in punctuate staining in cells stimulated by TNF-α (10 ng/ml) (FIG. 8D). Control cells (rat-IgG_{2b} only) were negative (FIG. 8A).

### Example 7

### Expression of PTX3 in Lung Tissues of Asthma Patients

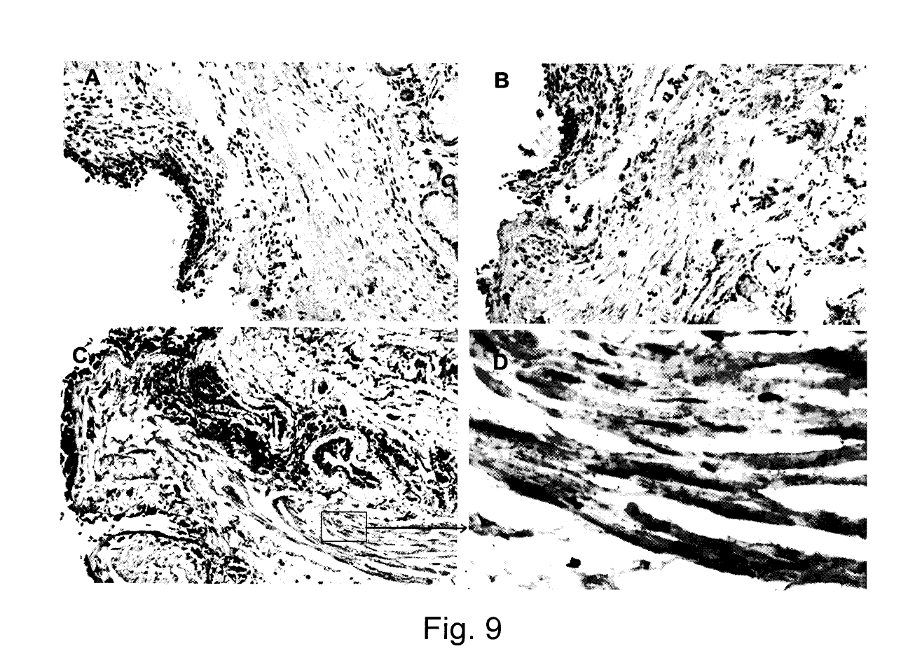

Lung tissue from subjects diagnosed with asthma was analyzed for expression of PTX3 by immunohistochemistry. Human airway sections were stained using the monoclonal antibody to PTX3 (MNB1) from a non-asthmatic subject (FIG. 9(A)), from asthmatic subjects (FIG. 9(B)) or matched slides with rat IgB_{2b} isotype matched control (FIGS. 9(C) & 9(D)). Tissue sections were then incubated with biotinylated anti-rat IgG (H+L) then processed with streptavidin-alkaline phosphatase stained with Fast Red. Immunohistochemistry was performed using lung biopsies sections from asthmatic and healthy volunteers in accordance with procedures approved by the Human Research Ethics Board of Laval University, Quebec, Canada. PTX3 rat mAb or control IgG2b (both at 2.5 μg/ml) were added and incubated overnight at 4° C. Biotin labeled rabbit anti-rat IgG (H+L) in 1:200 dilutions was added for 1 hr, processed with streptavidin-alkaline phosphatases, developed by fast red staining and counterstained with hematoxylin. Images were acquired with Olympus AX-70 microscope with a Photometrics PXL cooled CCD camera and Image-Pro Plus software (Carsen Group Inc., Markham, ON). E. High level of PTX3 expression in ASM cells bundle in allergic asthmatics compared to healthy control subjects. P<0.05 Specimens were scored as previously described (Fregonese L, et al, J Allergy Clin Immunol 2005; 115:1148) by three independent observers in a blinded manner using set scales between 0 and 3+: 0, absent staining or faint staining of an occasional ASM bundle only; 1+, faint staining of several ASM bundle: 2+, moderate intensity staining of most ASM bundle: and 3+, intense staining of most ASM bundle. Intense staining for PTX3 in samples from asthmatic subjects was mainly observed diffuse in the area of airway smooth muscle cells (ASM) (FIG. 9(B)). The staining was also detected in infiltrated inflammatory cells as well as in epithelial cells. However, the staining for PTX3 of non-asthmatic subjects was less intense and observed mainly around epithelial and ASM cells (FIG. 9(A)). Substitution of the first antibody with a matched rat-IgG_{2b} control eliminated the fast-red signal, demonstrating the specificity of the first antibody (FIGS. 9(C) & 9(D)).

### Example 8

### TNF-α-Induced PTX3 is Mediated Through Via MAPK (JNK and p42/p44 ERK) Pathways

One of the major downstream pathways for TNF-α-induced cell activation is MAPKs, which play an important role in cells for inflammatory response. Growth-arrested HASMC cells were left unstimulated (medium alone) or treated with 10 ng/ml TNF-α for 24 h, with or without pretreatment for 1 h with inhibitors of JNK (SP600125 50 nM), p42/p44 ERK (U-0126, 10 μM) and p38 MAPK (SB-203580, 10 μM).

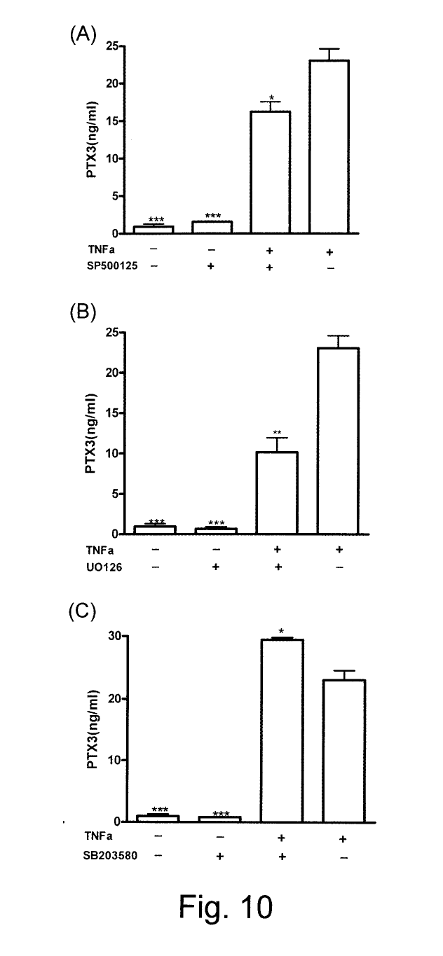

Treatment of HASMC with U-0126 (FIG. 10B) or SP600125 (FIG. 10A) before stimulation with TNF-α both caused a significant inhibition of PTX3. In contrast, treatment of HASMC with SB-203580 (FIG. 10C) had no effect on TNF-α induced PTX3 release by HASMC. These results indicate that JNK and p42/p44 ERK MAPK, but not p38, contribute to TNF-α-mediated release of PTX3 by HASMC.

One or more currently preferred embodiments of the invention have been described by way of example. The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

What is claimed is:

1. A method of detecting a risk for or the presence of asthma in a subject, comprising:
obtaining a biological sample from the subject;
testing the biological sample for presence of a pentraxin-3 (PTX3) polypeptide; and
correlating the presence of the PTX3 polypeptide in the biological sample with the risk for or the presence of asthma in the subject.

2. The method of claim 1, wherein the PTX3 polypeptide comprises an amino acid sequence that shares at least 80% sequence identity with SEQ ID NO: 1.

3. The method of claim 1, wherein the PTX3 polypeptide comprises SEQ ID NO: 1.

4. The method of claim 1, wherein the biological sample comprises human airway smooth muscle cells or serum or broncho-alveolar lavage fluid.

5. The method of claim 1, further comprising the step of comparing the presence of the PTX3 polypeptide in the biological sample with the presence of a PTX3 polypeptide in corresponding samples obtained from normal subjects lacking asthma.

6. The method of claim 1, wherein the assay comprises determining the presence of the PTX3 polypeptide in a plurality of biological samples obtained at successive time intervals from the subject.

7. The method of claim 6, wherein the assay comprises determining the presence of the PTX3 polypeptide in the biological sample at a time prior to treatment with an anti-asthma composition or an anti-inflammatory composition, and determining the presence of the PTX3 polypeptide in the biological sample at a time following treatment with the anti-asthma composition or the anti-inflammatory composition.

8. The method of claim 1, wherein the step of testing includes an immunologically based assay.

9. The method of claim 1, additionally comprising the steps of:
culturing a first portion of the biological sample in the presence of TNF-α;
testing the first cultured portion for the presence of the PTX3 polypeptide;
culturing a second portion of the biological sample in the absence of TNF-α;
testing the second cultured portion for the presence of the PTX3 polypeptide;
correlating the presence of the PTX3 polypeptide in the first cultured portion with the presence of the PTX3 polypeptide in the second cultured portion; and
wherein the presence of elevated levels of the PTX3 polypeptide in the first cultured portion is indicative of a risk for asthma in the subject.

10. A method of detecting a risk for or presence of an acute respiratory inflammatory response in a subject, comprising:
obtaining from the subject a biological sample comprising human airway smooth muscle cells;
testing the biological sample for the presence of a pentraxin-3 (PTX3) polypeptide; and
and correlating the presence of PTX3 in the biological sample with the risk for or presence of asthma in the subject.

11. The method of claim 9, wherein the biological sample comprises human airway smooth muscle cells or serum or broncho-alveolar lavage fluid.

12. The method of claim 9, further comprising the step of comparing the presence of the PTX3 polypeptide in the biological sample with the presence of the PTX3 polypeptide in corresponding samples from normal subjects lacking asthma.

13. The method of claim 9, wherein the assay comprises determining the presence of the PTX3 polypeptide in a plurality of biological samples obtained at successive time intervals from the subject.

14. The method of claim 13, wherein the assay comprises determining the presence of the PTX3 polypeptide in the biological sample at a time prior to treatment with an anti-asthma composition or an anti-inflammatory composition, and determining the presence of the PTX3 polypeptide in the biological sample at a time following treatment with the anti-asthma composition or the anti-inflammatory composition.

15. The method of claim 9, wherein the step of testing includes an immunologically based assay.

16. The method of claim 9, additionally comprising the steps of:
culturing a first portion of the biological sample in the presence of TNF-α;
testing the first cultured portion for the presence of the PTX3 polypeptide;
culturing a second portion of the biological sample in the absence of TNF-α;
testing the second cultured portion for the presence of the PTX3 polypeptide;
correlating the presence of the PTX3 polypeptide in the first cultured portion with the presence of the PTX3 polypeptide in the second cultured portion; and
wherein the presence of elevated levels of the PTX3 polypeptide in the first cultured portion is indicative of a risk for asthma in the subject.

17. Use of a pentraxin-3 (PTX3) polypeptide for detecting a risk for or the presence of asthma in a subject.

18. Use according to claim 17, wherein the PTX3 polypeptide comprises an amino acid sequence that shares at least 80% sequence identity with SEQ ID NO: 1.

19. Use according to claim 17, wherein the PTX3 polypeptide comprises SEQ ID NO: 1.
